(19) United States
(12) Patent Application Publication
(10) Pub. No.: US 2026/0117163 A1
(43) Pub. Date: Apr. 30, 2026

(54) Automated System for Signaling Cell Growth Events Using Continuous Sensor Data

(71) Applicant: Skroot Laboratory, Inc., Ames, IA (US)

(72) Inventors: Nigel F. Reuel, Ames, IA (US); Subhanwit Roy, Ames, IA (US); Aidan Shervheim, Ames, IA (US); Samuel Rothstein, Ames, IA (US); Cameron Greenwalt, Oakdale, MN (US)

(73) Assignee: Skroot Laboratory, Inc., Ames, IA (US)

(21) Appl. No.: 19/341,946

(22) Filed: Sep. 26, 2025

Related U.S. Application Data
(60) Provisional application No. 63/714,520, filed on Oct. 31, 2024.

(51) Int. Cl. C12M 1/36; C12M 1/12; C12M 1/34

(52) U.S. Cl. CPC C12M 41/48; C12M 37/00; C12M 41/36

(57) ABSTRACT

An automated system for signaling cell growth events using continuous sensor data includes at least one sensor associated with a vessel configured for use in growth of cells, the sensor providing continuous sensor data associated with cell growth, a reader in operative communication with the sensor to collect the continuous sensor data, and a computational unit operatively connected to the reader, wherein the computational unit is configured to receive as input the continuous sensor data and calibration data associated with process conditions, the computational unit further configured to perform signal processing on the continuous sensor data in order to predict at least one of future critical events and generate insights about unexpected behavior.

### PRIORITY STATEMENT

This application claims priority to U.S. Provisional Patent Application No. 63/714,520, filed Oct. 31, 2024, entitled Automated System for Signaling Cell Growth Events Using Continuous Sensor Data, hereby incorporated by reference in its entirety.

### FIELD OF THE INVENTION

The present invention relates to monitoring cell growth within a vessel. More particularly, but not exclusively, the present invention relates to an automated system and methods for signaling cell growth events using continuous sensor data.

### BACKGROUND

Various approaches to monitoring a cell culture are discussed herein. It is to be understood that the relationship between these different approaches may not be apparent to one skilled in the art without the benefit of this disclosure.

U.S. Published Patent Application No. 2022/024122 describes using wireless sensors, such as resonant sensors, to track cell growth inside a vessel. U.S. Pat. No. 11,636,755 describes capturing a raw analog signal from the sensor with a wireless reader which converts the raw analog signal to digital form and passes it on to a computational unit (laptop, tablet, or other computational hardware) which generates a plot of the change in resonant frequency versus time.

Although such an approach is advantageous, problems remain. For example, although the plot as a proxy for cell growth is provided, it requires a trained operator to monitor it intermittently and make decisions, such as when the cell culture is ready to be harvested, based on their judgment. This human-driven approach increases labor costs and introduces the potential for subjectivity and error.

More conventional cell-growth tracking technologies may involve intermittent optical density analysis or cell counting systems, which require technician intervention and run high risks of contamination. Thus, continuous sensor systems are advantageous.

Thus, problems remain. For example, currently there is no existing solution for generating automated machine-driven insights regarding critical future events or divergent behavior from real-time raw continuous sensor data. What is needed is a new approach of generating automated insights specific to future or unexpected cell growth stages from continuous sensor data.

### SUMMARY

Therefore, it is a primary object, feature, or advantage of the present invention to improve over the state of the art.

It is a further object, feature, or advantage of the present invention to provide continuous monitoring of cell growth, improving data density over periodic sampling.

It is a still further object, feature, or advantage of the present invention to automate the analysis of continuous sensor data using use-case-specific calibration information.

Another object, feature, or advantage is to predict the timing of future critical events, such as when the cell culture will be ready for harvest.

Yet another object, feature, or advantage is to detect unexpected events, such as contamination, that indicate abnormal or divergent cell growth behavior.

It is a further object, feature, or advantage of the present invention to reduce reliance on the judgment of experienced scientists by implementing an automated decision-making process.

It is a still further object, feature, or advantage of the present invention to lower labor costs by reducing the need for manual oversight in monitoring cell culture status.

Another object, feature, or advantage is to increase objectivity in decision-making by setting reliable, automated standards for detecting critical events in cell culture.

Yet another object, feature, or advantage is to enable faster response times by providing real-time warnings to operators when critical events are predicted.

It is a further object, feature, or advantage of the present invention to decrease the time to take corrective action after critical events are detected.

It is a still further object, feature, or advantage of the present invention to simplify the operation of monitoring systems by reducing the technical expertise required of the operator.

It is a further object, feature, or advantage of the present invention to eliminate the need for technician intervention by providing fully automated monitoring of cell growth.

It is a still further object, feature, or advantage of the present invention to reduce the risk of contamination by minimizing manual handling through continuous sensor systems.

Another object, feature, or advantage is to outperform intermittent optical density analysis and cell counting systems by delivering continuous, real-time data and analysis without the need for technician oversight.

Yet another object, feature, or advantage is to surpass existing technologies such as capacitance probes by generating predictions for critical future events, rather than only identifying them after they occur.

It is a further object, feature, or advantage of the present invention to provide real-time adaptability by continuously refining predictions based on live sensor data, unlike current methods that rely solely on static calibration data.

It is a still further object, feature, or advantage of the present invention to generate actionable insights not only for pre-defined conditions but also for unexpected or divergent cell culture behavior.

Another object, feature, or advantage is to fill the gap left by existing technologies, which do not offer real-time future event prediction or anomaly detection from continuous data.

Yet another object, feature, or advantage is to enable automated machine-driven decision-making based on continuous sensor data, thus reducing human error and subjectivity.

It is a further object, feature, or advantage of the present invention to allow real-time adaptation of cell culture management based on continuously updated insights into the cell growth status.

It is a still further object, feature, or advantage of the present invention to offer an unprecedented level of automation and intelligence in cell growth monitoring by predicting critical events and detecting anomalies before they occur.

Another object, feature, or advantage is to enhance efficiency in bioprocessing by reducing the time and labor required for manual monitoring and intervention.

Yet another object, feature, or advantage is to improve the reliability of cell culture outcomes by providing more precise, real-time data and/or electronic communication to allow for control over cell growth stages and reducing the risk of errors in feeding schedules or harvest timing.

It is a further object, feature, or advantage of the present invention to increase the scalability of biomanufacturing by automating continuous monitoring and predictive analysis across various cell cultures.

One or more of these and/or other objects, features, or advantages of the present invention will become apparent from the specification and claims that follow. No single embodiment need provide each and every object, feature, or advantage. Different embodiments may have different objects, features, or advantages. Therefore, the present invention is not to be limited to or by any objects, features, or advantages stated herein.

According to one aspect, an automated system leverages continuous sensor data, along with use-case-specific calibration information, to derive real-time insights into the monitored cell culture's status, including (i) predicting the timing of future critical events (such as time to be ready for harvest) and (ii) detecting unexpected events that indicate abnormal or divergent cell growth behavior (such as possible contamination). Thereby, enabling objective standards for reliably triggering critical insights (e.g., pushed warnings to operators) into an active cell culture and decrease the time to take action after critical events, while reducing the technical expertise required of the operator, thus lowering labor costs.

According to another aspect, an automated system for signaling cell growth events using continuous sensor data is provided. The system includes at least one sensor associated with a vessel configured for use in growth of cells, the sensor providing continuous sensor data associated with cell growth. The system further includes a reader in operative communication with the sensor to collect the continuous sensor data. The system further includes a computational unit operatively connected to the reader, wherein the computational unit is configured to receive as input the continuous sensor data and calibration data associated with process conditions, the computational unit further configured to perform signal processing on the continuous sensor data in order to predict future critical events. The system may further include a display operatively connected to the computational unit and wherein the computational unit is configured to display output based on the future critical events predicted by the computational unit. The computational unit may be configured to electronically send notifications regarding the future critical events to a device associated with a user such as a scientist, technician, or other operator or stakeholder. The computational unit may be further configured to perform signal processing on the continuous sensor data to generate insights about unexpected behavior. The system may include a display operatively connected to the computational unit and wherein the computational unit is configured to display output based on the insights about the unexpected behavior generated by the computational unit. The computational unit may be further configured to electronically send notifications regarding the insights about the unexpected behavior to a device associated with a stakeholder. The unexpected behavior may be associated with contamination. The signal processing may be performed using at least one machine learning algorithm and/or at least one deterministic algorithm. The calibration data may include at least one of vessel type, cell type, media, seeding conditions, cell growth performance, sensor response, metabolite levels, cell count/viability, and assays from prior runs. The sensor may be a resonant sensor.

According to another aspect, a method for automated signaling of cell growth events may include the steps of providing at least one sensor for use within a vessel configured for use in cell growth, the sensor providing continuous sensor data associated with the cell growth. The method may further provide for collecting the continuous sensor data from the sensor using a reader in operative communication with the at least one sensor. The method may further provide for receiving, by a computational unit, the continuous sensor data and calibration data associated with process conditions. The method may further include performing signal processing on the continuous sensor data, by the computational unit, to predict future critical events related to cell growth. The method may further include displaying output, based on the future critical events predicted by the computational unit, on a display operatively connected to the computational unit. The method may further include sending notifications electronically, by the computational unit, to a device associated with a user, the notifications being based on the predicted future critical events. The method may further include performing signal processing on the continuous sensor data, by the computational unit, to generate insights about unexpected behavior during cell growth. The method may further include displaying, on a display operatively connected to the computational unit, output based on the insights about unexpected behavior generated by the computational unit. The method may further include sending notifications electronically, by the computational unit, to a device associated with a user, the notifications being based on the insights about unexpected behavior.

### BRIEF DESCRIPTION OF THE DRAWINGS

Illustrated embodiments of the disclosure are described in detail below with reference to the attached drawing figures, which are incorporated by reference herein.

FIG. 1 is a block diagram of a system which provides for the transformation of the raw signal from the resonant sensor to the automated insights with the help of application-specific calibration data.

FIG. 2 illustrates an example of time prediction of the occurrence of a critical future event such as forecasting when the cells will be optimally ready to be harvested.

FIG. 3 illustrates automated insights triggered on the occurrence of unexpected events that indicate abnormal or divergent behavior where normalcy is pre-established using calibration data.

FIG. 4 is a flowchart illustrating one example of a method.

### DETAILED DESCRIPTION

The present disclosure provides for generating automated machine-driven insights regarding critical future events or divergent behavior from real-time raw continuous sensor data.

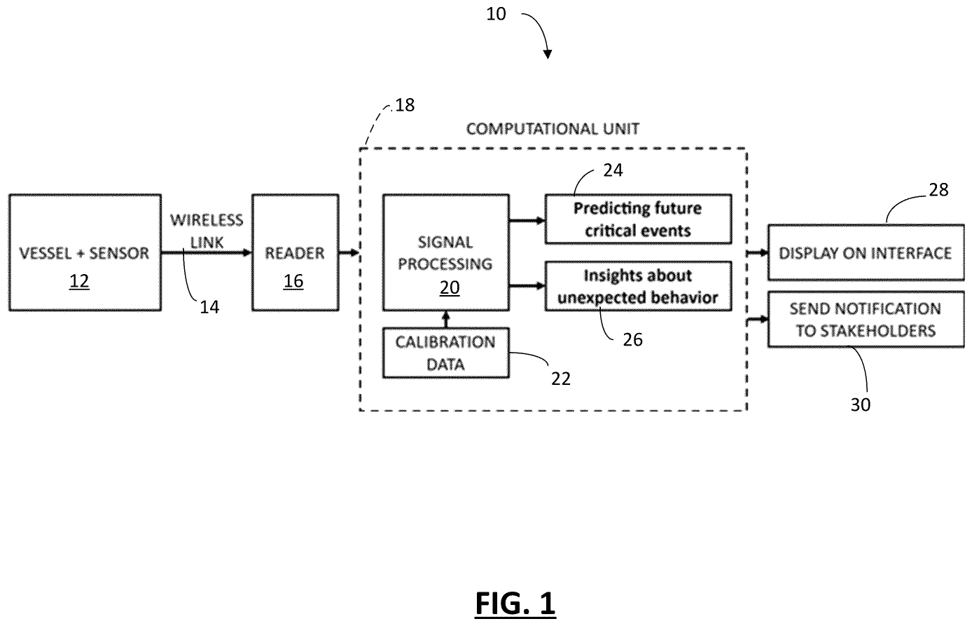

FIG. 1 illustrates one example of an automated system for signaling cell growth events. A system 10 includes a vessel 12 with an associated sensor. The vessel 12 may be of any number of types of vessels including any type of bioreactor containers, flasks, bags, or industrial size containers. The vessels may be of numerous types of materials provided that they do not interfere with the sensor or transmission of continuous sensor data over the wireless link 14. The sensor may be a resonant sensor configured to provide for continuous monitoring of cell growth parameters. The sensor may be adhered or otherwise attached to an inner or outer surface of the vessel.

The system 10 further includes a reader 16 in operative communication with the sensor attached to the vessel 12 such as over a wireless link 14. The reader 16 is configured to collect the continuous sensor data and communicate the continuous sensor data to a computational unit 18 14. The computational unit 18 includes a signal processing block 20 which is configured to process the continuous sensor data in conjunction with calibration data 22. In some embodiments, at least a portion of the signal processing block 20 may be implemented in the reader 16. Although the computational unit and the reader are shown as separate blocks, it is to be understood that aspects of these blocks may be integrated or otherwise work together to perform operations such as reading and signal processing. The calibration data 22 may include factors such as vessel type, cell type, media composition, seeding conditions, and historical cell growth performance. Data associated with historical cell growth performance may include sensor response, metabolite levels, cell count/viability, assays, or other data. This calibration data 22 enhances the accuracy of the signal processing performed by the computational unit 18.

The computational unit 18 and its signal processing block 20 may use machine learning algorithms or other signal processing techniques to analyze the sensor data and generate predictions about future critical events 24, such as the optimal time for harvesting the cell culture or potential issues like contamination, In some embodiments, the computational unit 18 is further configured to detect unexpected or divergent behavior in the cell culture, such as abnormal growth patterns or metabolic shifts, and generate corresponding insights unexpected behavior 26.

The calibration data may be continuously augmented and improved through continuous machine learning as the process is iteratively run.

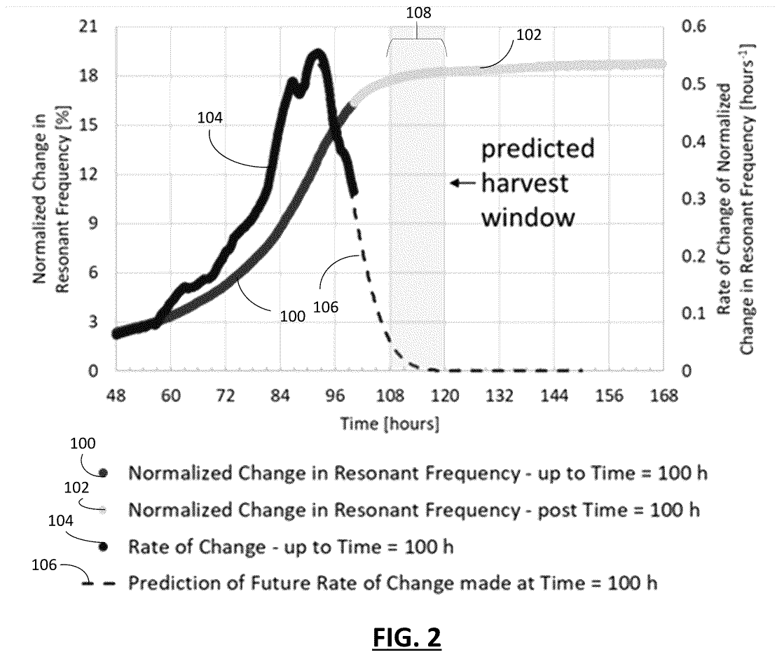

FIG. 2 is a plot illustrating a time prediction of the occurrence of a critical future event such as forecasting when the cells will be optimally ready to be harvested. The system makes the prediction by processing the sensor response with reference to thresholds determined from use-case specific calibration data. This insight may be used to avoid early or late harvests and ensure that cells are harvested at their optimal yield. In addition, it allows a cell manufacturing facility to prepare in advance to make the harvest on time.

FIG. 2 shows the sensor response (normalized change in resonant frequency 100) and the corresponding rate of change 104 of the same K562 culture. The data received up to time=100 h (as shown with plot lines 100 and 104) is used to predict when the cells will enter stationary phase, which in this particular context determines the start of the optimal harvest window 108. For this particular use case, a Gaussian fit around the critical growth rate is used, along with the calibration information consisting of a predefined threshold rate of 0.01 units/hour and the harvest window span being 12 hour wide around the threshold. This results in a predicted harvest window 108 between approximately 108 to 120 hours. The sensor response post 100 hours (as shown with line 102) shows the predicted harvest window indeed corresponds with the stationary phase of cell growth. The prediction would continue to be refined as more incoming data was processed in real-time beyond the 100 hour mark.

This specific use-case can be applied to the growth and monitoring of therapeutic cells. These therapies need to be expanded in a bioreactor to reach sufficient dose, but if they are left to grow too long their therapeutic potency is decreased. There is an optimal window in which to harvest, and this algorithm approach can predict where that window is based on a few calibration runs with the specific cell and vessel type.

The method described may be performed using software instruction. In addition, any number of machine learning methods may be provided. Any number of different types of machine learning algorithms or combinations of machine learning algorithms may be used. For example, continuous sensor data may be monitored and used to generate real-time predictions regarding critical cell growth event using, Recurrent Neural Networks (RNNs), including Long Short-Term Memory (LSTM) networks, may be employed to capture and model time-dependent relationships in the sensor data, providing predictions about future events such as the entry into the stationary phase based on historical trends. Additionally, Bayesian Inference techniques may be used to continuously update the predictions as new sensor data becomes available, refining the likelihood of certain critical events in real time. Of course, any number of different machine learning methods may be used as may be appropriate for a particular application or use.

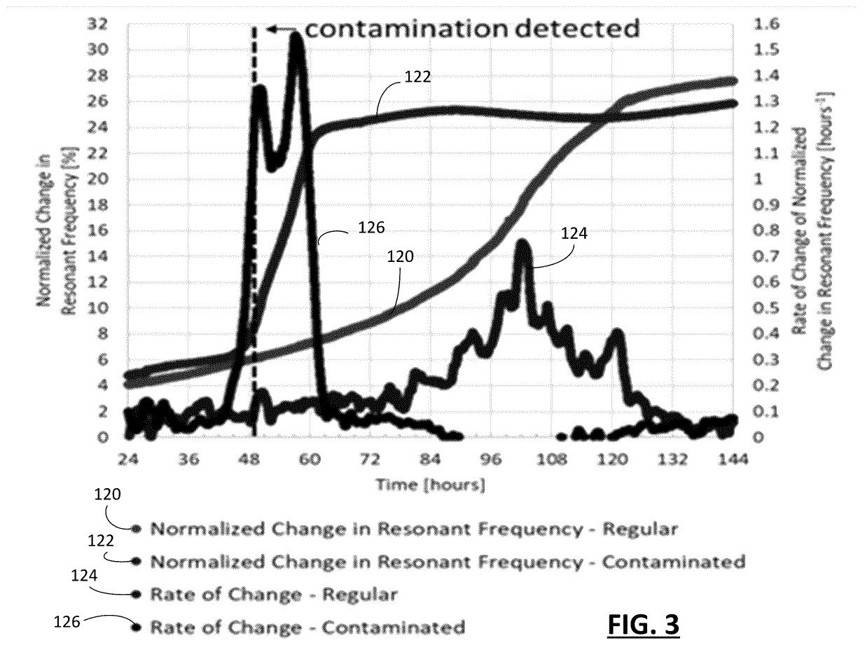

FIG. 3 is a plot showing automated insights triggered on the occurrence of unexpected events that indicate abnormal or divergent behavior where normalcy is pre-established using calibration data. Examples include slow-growing cell cultures, contamination events and mass cell deaths. Knowledge of such events can enable the cell manufacturing facility to quickly intervene or abort the divergent cell culture, thus potentially freeing up time and resources. In particular, FIG. 3 illustrates the sensor response 120, 122 and corresponding rate of change 124, 126 of two CHO cell cultures grown simultaneously under identical conditions. One of the cultures 122 was contaminated leading to a premature and sudden increase in the sensor response. This event was captured through the rate of change of the sensor response 126 crossing the externally set threshold of 1 unit/hour (for 10 successive sweeps, at time=48.9 hours), which is more than 25% larger than the critical growth rate reached by the uncontaminated culture at a later time (<0.8 units/hour). In the absence of this automated insight, one would have to depend on the subjective judgment of a scientist looking at the sensor response and deciding if it was indeed a case of contamination or, simply, fast cell growth. Also, the timely nature of the automated insight (e.g., pushed notification to an operator via email or text or other message) could enable avoiding delays in discarding the vessel, thus decreasing the risk of the contaminant potentially spreading to and disrupting other nearby cell culture vessels.

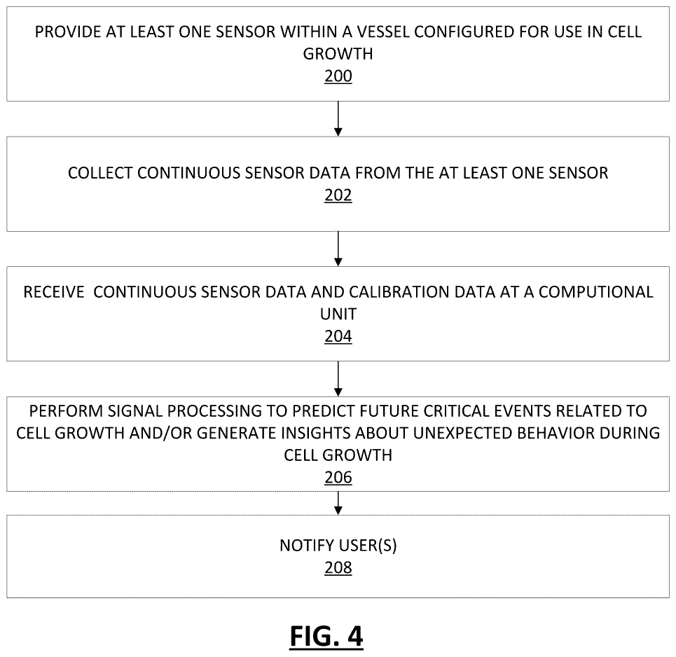

FIG. 4 illustrates one example of a method for automated signaling of cell growth events. In step 200, at least one sensor is provided and may be installed, attached within a vessel which is configured for use in cell growth. In step 202, continuous sensor data is collected from the at least one sensor during cell growth. In step 204, continuous sensor data is received at a computational unit which also receives calibration data. Next, in step 206, signal processing is performed to predict future critical events related to cell growth and/or insights are generated about unexpected behavior during cell growth.

The future critical events related to cell growth may include the transition from the exponential growth phase to the stationary phase, indicating the optimal harvest window for the cell culture, this may include predictions of the start time for the harvest window, the end time for the harvest window or both a start time and an end time for the harvest window. Other examples of critical events could include reaching the desired cell density or metabolite concentration, or the depletion of critical nutrients in the growth medium, requiring intervention such as nutrient feeding or media replacement. The insights about unexpected behavior during cell growth may include, without limitation, detection of contamination, indicated by abnormal changes in the sensor data, or unanticipated shifts in growth rate that could signal issues like poor nutrient absorption or genetic instability in the culture. These insights could also include the identification of anomalies in the metabolic activity of the cells, such as an unexpected buildup of by-products, or deviations from standard growth patterns that might suggest equipment malfunction or environmental stressors. By providing real-time predictions and insights, the system enables proactive decision-making and timely interventions, ensuring the cell culture remains on track for optimal growth and harvest.

In step 208, one or more users may be notified. A user can be any stakeholder permitted access to view information on a display and/or receive electronic notifications about a cell culture. Users may be notified in different ways. For example, users may be alerted by information displayed on a display associated with the computational unit. Users may be alerted via email. Users may be alerted via text messages. Users may be alerted via app notifications. It is contemplated that users may be notified in any meaningful way. Such notifications can be performed in any number of instances. For example, the notifications can be made once a harvest window is predicted, notifications can occur when the time to perform a harvest is near, when there is a change in when the harvest window is predicted to occur, when contamination is detected, when unclassified anomalies occur, or at any number of other times. The alert may include textual information indicative of the reason for the alert, plots such as or similar to those shown in FIG. 1 and FIG. 2, or any other information which may assist the user in identifying or understanding the issue and potential actions to take. It is further contemplated that the user need not be an individual but may hardware or software associated with the device which may, for example, log the occurrence of the alert, generate a report regarding the alert, or take other automated action such as automatically controlling environmental conditions, initiating an automated feeding adjustment, initiate an automated harvest process, initiate an automated contamination mitigation process such as an automated sterilization or isolation protocol, or other automated actions. It is further contemplated that any number of users may be notified of events.

Therefore, methods and systems have been shown and described which provide for continuous monitoring of cell growth, such as with resonant sensors, thereby improving data density over periodic sampling of cell cultures. Moreover, the present disclosure has shown and described an automated system that leverages continuous sensor data, along with use-case-specific calibration information, to derive real-time insights into the monitored cell culture's status, including (i) predicting the timing of future critical events (such as time to be ready for harvest) and (ii) detecting unexpected events that indicate abnormal or divergent cell growth behavior (such as possible contamination). Thereby, this innovation can be used to enable objective standards for reliably triggering critical insights (e.g., pushed warnings to operators or other users or stakeholders) into an active cell culture and decrease the time to take action after critical events, while reducing the technical expertise required of the operator, thus lowering labor costs.

The invention is not to be limited to the particular embodiments described herein. In particular, the invention contemplates numerous variations in the types of vessels, types of sensors used for continuous monitoring, type of cell cultures, number of sensors, algorithms used in processing the continuous data, the type of calibration data used, the manner in which results of the analysis are conveyed to users, and any number of other options, variations, and alternatives. The foregoing description has been presented for purposes of illustration and description. It is not intended to be an exhaustive list or limit any of the invention to the precise forms disclosed. It is contemplated that other alternatives or exemplary aspects are considered included in the invention. The description is merely examples of embodiments, processes, or methods of the invention. It is understood that any other modifications, substitutions, and/or additions can be made, which are within the intended spirit and scope of the invention.

What is claimed is:

1. An automated system for signaling cell growth events using continuous sensor data, comprising:
at least one sensor associated with a vessel configured for use in growth of cells, the at least one sensor providing continuous sensor data associated with cell growth;
a reader in operative communication with the at least one sensor to collect the continuous sensor data; and
a computational unit operatively connected to the reader, wherein the computational unit is configured to receive as input the continuous sensor data and calibration data associated with process conditions, the computational unit further configured to perform signal processing on the continuous sensor data in order to predict future critical events.

2. The automated system of claim 1 further comprising a display operatively connected to the computational unit and wherein the computational unit is configured to display output based on the future critical events predicted by the computational unit.

3. The automated system of claim 1 wherein the computational unit is further configured to electronically send notifications regarding the future critical events to a device associated with a user.

4. The automated system of claim 1 wherein the computational unit is further configured to generate insights about unexpected behavior.

5. The automated system of claim 4 further comprising a display operatively connected to the computational unit and wherein the computational unit is configured to display output based on the insights about the unexpected behavior generated by the computational unit.

6. The automated system of claim 4 wherein the computational unit is further configured to electronically send notifications regarding the insights about the unexpected behavior to a device associated with a user.

7. The automated system of claim 4 wherein the unexpected behavior is associated with contamination.

8. The automated system of claim 1 wherein the signal processing is performed using at least one of a machine learning algorithm and a deterministic algorithm.

9. The automated system of claim 1 wherein the calibration data comprises at least one of vessel type, cell type, media, seeding conditions, cell growth performance, sensor response, metabolite levels, cell count/viability, and assays from prior runs.

10. The automated system of claim 1 wherein the at least one sensor is a resonant sensor.

11. A method for automated signaling of cell growth events in a cell culture, comprising:
provide at least one sensor for use within a vessel configured for use in cell growth, the at least one sensor providing continuous sensor data associated with the cell growth;
collecting the continuous sensor data from the at least one sensor using a reader in operative communication with the at least one sensor;
receiving, by a computational unit, the continuous sensor data and calibration data associated with process conditions; and
performing signal processing on the continuous sensor data, by the computational unit, to predict future critical events related to cell growth.

12. The method of claim 11 further comprising displaying output, based on the future critical events predicted by the computational unit, on a display operatively connected to the computational unit.

13. The method of claim 11 further comprising sending notifications electronically, by the computational unit, to a device associated with a user, the notifications being based on the predicted future critical events.

14. The method of claim 11 further comprising performing generating insights about unexpected behavior during cell growth.

15. The method of claim 14 further comprising displaying, on a display operatively connected to the computational unit, output based on the insights about unexpected behavior generated by the computational unit.

16. The method of claim 14 further comprising sending notifications electronically, by the computational unit, to a device associated with a user, the notifications being based on the insights about unexpected behavior.

17. The method of claim 14, wherein the unexpected behavior is associated with contamination of the cell culture.

18. The method of claim 11, wherein the signal processing is performed using at least one of a machine learning algorithm and a deterministic algorithm.

19. The method of claim 11, wherein the calibration data comprises at least one of vessel type, cell type, media, seeding conditions, cell growth performance, sensor response, metabolite levels, cell count/viability, and assays from prior runs.

20. The method of claim 11, wherein the at least one sensor is a resonant sensor.
